(19) United States
(12) Patent Application Publication
(10) Pub. No.: US 2022/0019952 A1
(43) Pub. Date: Jan. 20, 2022

(54) FAIR SCHEDULING OF NURSES IN RESOURCE CONSTRAINED SETTINGS

(71) Applicant: Siemens Medical Solutions USA, Inc., Malvern, PA (US)

(72) Inventors: Ahmet Tuysuzoglu, Jersey City, NJ (US); Jongeun Kim, Minneapolis, MN (US)

(21) Appl. No.: 17/303,611

(22) Filed: Jun. 3, 2021

Related U.S. Application Data
(60) Provisional application No. 63/052,065, filed on Jul. 15, 2020.

(51) Int. Cl. G06Q 10/06; G16H 40/20; G06Q 10/04

(52) U.S. Cl. CPC G06Q 10/06314; G06Q 10/04; G16H 40/20

(57) ABSTRACT

Systems and methods for determining a schedule assigning individuals to shifts are provided. A plurality of constraints for scheduling individuals to shifts is received. The plurality of constraints comprise one or more hard constraints and one or more soft constraints. A schedule assigning the individuals to the shifts is determined that 1) satisfies the one or more hard constraints and 2) distributes deviations from the one or more soft constraints across the determined schedule. The determined schedule is output. In one embodiment, the individuals are nurses.

This application claims the benefit of U.S. Provisional Application No. 63/052,065, filed Jul. 15, 2020, the disclosure of which is herein incorporated by reference in its entirety.

### TECHNICAL FIELD

The present invention relates generally to a scheduling system for automatically scheduling individuals to shifts, and in particular to a scheduling system for the fair scheduling of nurses in resource constrained settings.

### BACKGROUND

With the current emphasis on value-based healthcare, patient outcomes and key performance indicators (KPIs) such as, e.g., the occurrence of adverse events, mortality rates, and patient experience scores, are tracked. Such patient outcomes and KPIs directly affect reimbursement amounts and hence it is important for healthcare providers to achieve favorable patient outcomes and KPIs for financial viability. It has been found that a satisfied and adequately staffed nurse workforce highly correlates with positive patient outcomes and KPIs. However, a shortage of nurses has been forecasted in the near future. The lack of a satisfied, adequately staffed nurse workforce can be alleviated by scheduling nurses to shifts that address their preferences, unavailabilities, paid time off requests, maximum working hours, and fairness. The problem of identifying an optimal assignment of nurses to shifts is commonly referred to as the nurse scheduling problem.

Various conventional approaches have been proposed for addressing the nurse scheduling problem. However, such conventional approaches do not incorporate seniority-based considerations and the resulting schedule is not well balanced in terms of resources and preferences.

### BRIEF SUMMARY OF THE INVENTION

In accordance with one or more embodiments, systems and methods for determining a schedule assigning individuals to shifts are provided. A plurality of constraints for scheduling individuals to shifts is received. The plurality of constraints comprise one or more hard constraints and one or more soft constraints. A schedule assigning the individuals to the shifts is determined that 1) satisfies the one or more hard constraints and 2) distributes deviations from the one or more soft constraints across the determined schedule. The determined schedule is output. In one embodiment, the individuals are nurses.

In one embodiment, the one or more soft constraints comprises preferences for desirable and undesirable shifts or days for one or more of the individuals. The schedule is determined by determining a maximally achievable preference score for the one or more individuals for the preferences and minimizing a deviation of the determined schedule from the maximally achievable preference score for the desirable shifts or days and minimizing a deviation of the determined schedule from the undesirable shifts or days.

In one embodiment, the schedule is determined by optimizing one or more slack variables representing deviations of a schedule from the one or more soft constraints. One or more cost functions may be defined for optimizing the one or more slack variables as one or more piecewise linear cost functions. A multi-objective optimization problem may be optimized to identify a solution for each objective of the multi-objective optimization problem and the determined schedule may be selected from the identified solutions. In one embodiment, the multi-objective optimization problem is optimized by lexicographically solving the multi-objective optimization problem by assigning an order of importance to each objective of the multi-objective optimization problem and sequentially solving each objective based on the assigned order of importance. In another embodiment, the multi-objective optimization problem is optimized by weighting each objective of the multi-objective optimization problem and optimizing the multi-objective optimization problem based on the weights.

In one embodiment, the plurality of constraints comprises one or more of: an assignment of one or more of the individuals to one or more of the shifts; one or more of the shifts that one or more of the individuals cannot be assigned; a maximum number of shifts in a day that one or more of the individuals can be assigned; a maximum number of consecutive days one or more of the individuals can be assigned a shift; two or more succeeding shifts that one or more of the individuals cannot be assigned a shift; an assignment of one or more of the individuals having a certain set of skills or certificates to one or more of the shifts; an assignment of one or more of the individuals with a certain seniority or title to one or more of the shifts; a number of hours one or more of the individuals are assigned; a minimum number of the individuals assigned to one or more of the shifts; an assignment of one or more of the individuals to weekend shifts; and an assignment of one or more of the individuals to their preferred shifts or days.

These and other advantages of the invention will be apparent to those of ordinary skill in the art by reference to the following detailed description and the accompanying drawings.

### BRIEF DESCRIPTION OF THE DRAWINGS

FIG. 1 shows a method for automatically determining a schedule assigning individuals to shifts, in accordance with one or more embodiments;

FIG. 2 shows a table of constraints used for experimentally validating embodiments described herein;

FIG. 3A shows a graph illustrating a distribution of excess or missing number of nurses for each shift across resource levels to compare embodiments described herein with various other approaches;

FIG. 3B shows a graph illustrating a ratio of shifts that have no deviation from their target number of nurses to compare embodiments described herein with various other approaches;

FIG. 4A shows a graph illustrating a distribution of deviation from target working hours to compare embodiments described herein with various other approaches;

FIG. 4B shows a graph illustrating the ratio of nurses that have no deviation from their target working hours to compare embodiments described herein with various other approaches;

FIG. 5A shows a graph illustrating a deviation from a surrogate maximum achievable score to compare embodiments described herein with various other approaches;

FIG. 5B shows a graph illustrating no-deviation ratio to compare embodiments described herein with various other approaches; and

FIG. 6 shows a high-level block diagram of a computer that may be used to implement one or more embodiments.

### DETAILED DESCRIPTION

Embodiments described herein provide for a scheduling system for the automatic scheduling of nurses (or other individuals) to shifts in which constraints for the scheduling are defined to be hard constraints or soft constraints. The scheduling system provides for fair scheduling of nurses by balancing or distributing deviations from the soft constraints across the schedule. The scheduling system applies a modeling concept to assign nurses to their preferred shifts while considering different levels of priority based on seniority and other factors, such as, e.g., the type of contract and satisfaction levels.

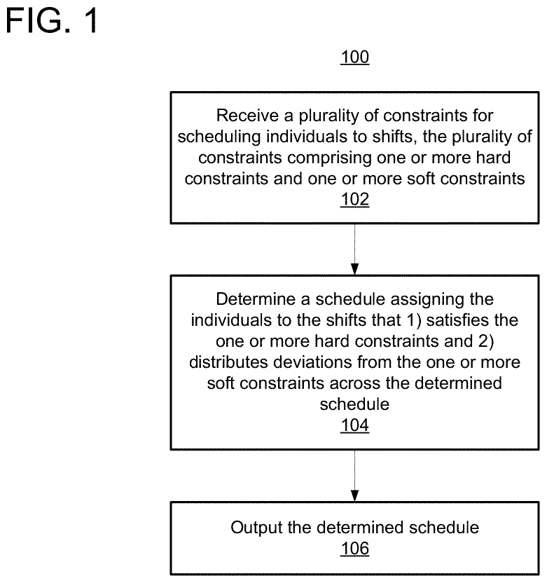

FIG. 1 shows a method 100 for automatically determining a schedule assigning individuals to shifts, in accordance with one or more embodiments. The steps of method 100 may be performed by automatic scheduling system implemented by one or more suitable computing devices, such as, e.g., computer 702 of FIG. 7.

At step 102, a plurality of constraints for scheduling individuals to shifts are received. The plurality of constraints comprise one or more hard constraints and one or more soft constraints. The hard constraints are non-negotiable requirements for scheduling the individuals to the shifts. The soft constraints are negotiable preferences for scheduling the individuals to the shifts. The shifts are periods of time at which the individuals are scheduled to work. The constraints may be received from users, such as, e.g., the individuals and/or an entity managing the scheduling (e.g., a manager). In some embodiments, one or more of the constraints are predefined as being hard constraints. In other embodiments, one or more of the constraints are customizable constraints that are defined by the users as being a hard constraint or a soft constraint.

In one embodiment, the individuals are nurses and the shifts include, for example, a morning shift, an evening shift, and an overnight shift. Embodiments described herein will be described where the individuals are nurses in accordance with this embodiment. However, it should be understood that the present invention is not so limited and the embodiments described herein may be applied to schedule any individual (e.g., employee) to shifts.

The constraints may comprise any suitable constraint for scheduling the nurses to the shifts. In one embodiment, the constraints comprise one or more of the following constraints: 1) the assignment of particular nurses to particular shifts, 2) particular shifts that particular nurses cannot be assigned, 3) a certain maximum number of shifts in a day that particular nurses can be assigned, 4) a certain maximum number of consecutive days particular nurses can be assigned shifts, 5) two or more particular succeeding shifts that particular nurses cannot be assigned, 6) the assignment of particular nurses having a certain set of skills, certificates, etc. to particular shifts, 7) the assignment of particular nurses with a certain seniority, title, etc. (e.g., a charge nurse) to particular shifts, 8) a particular number of hours particular nurses are assigned, 9) a minimum number of nurses assigned to particular shifts, 10) the assignment of particular nurses to weekend shifts, and 11) assignment of nurses to their preferred shifts or days. Other constraints may also be utilized for scheduling the nurses to the shifts.

At step 104 of FIG. 1, a schedule assigning the individuals to the shifts is determined that 1) satisfies the one or more hard constraints and 2) distributes deviations from the one or more soft constraints across the determined schedule.

Formally, the assignment of nurse n∈N to shift s∈S on day d∈D is denoted by a binary decision variable x_{n,d,s}, where N, S, and D represent the sets of nurses, shifts, and days, respectively. For constraints that are customizable as being a hard constraint or a soft constraint, a slack variable [symbol] indexes representing the deviation from the constraint in a schedule is introduced, where name represents the type of the associated constraint and indexes represents a set of indexes corresponding to the constraint. Unless otherwise specified, a slack variable is continuous and nonnegative such that [symbol] _{indexes}^{name}≥0. Where the customizable constraint is defined to be a hard constraint, the corresponding slack variable will be zero. The constraints identified above with respect to step 102 will be formally defined in accordance with the following functions.

Constraint 1: The assignment of particular nurses to particular shifts may be defined as being hard constraints as follows:

x_{n,d,s}=1 (Equation 1)

where (n,d,s)∈PreAssignments and PreAssignments is a set of indexes of pre-assignments.

Constraint 2: Particular shifts that particular nurses cannot be assigned (e.g., due to requests for time off) may be defined as being hard constraints as follows:

x_{n,d,s}=0, (n,d,s)∈Off Requests (Equation 2)

where OffRequests is a set of indexes of shifts that nurses cannot be assigned to.

Constraint 3: A certain maximum number of shifts in a day that particular nurses can be assigned may be customized as being a hard constraint or a soft constraint as follows:

$${\sum }_{s ∈ S} {x}_{n , d , s} \leq m a x Shift {s}_{n , d} + {ℓ}_{n , d}^{maxShifts} \qquad ( Equation 3 )$$

where n∈N, d∈D and where maxShifts_{n,d} is a user defined maximum number of shifts allowed for nurse n on day d.

Constraint 4: A certain maximum number of consecutive days particular nurses can be assigned shifts may be customized as being a hard constraint or a soft constraint as follows:

$${{\sum }_{i = d}}^{d + m C {D}_{n}} {y}_{n , i} \leq m C {D}_{n} + {ℓ}_{n , d 1 , s 1 , d 2 , s 2}^{n o F o l l o w} \\ {y}_{n , d} \geq \frac{1}{\vert S \vert} {\sum }_{s ∈ S} {x}_{n , d , s} \qquad ( Equation 4 )$$

where n∈N and d∈D. y_{n,d}∈{0,1} is an indicator variable denoting whether a nurse worked on day d regardless of the number of shifts worked. mCDₙ is a user defined variable that indicates the maximum number of consecutive days nurse n may be assigned.

Constraint 5: Two or more particular succeeding shifts that nurses cannot be assigned may be customized as being a hard constraint or a soft constraint as follows:

x_{n,d1,s1}+x_{n,d2,s2}≤1+ [symbol] _{n,d1,s1,d2,s2}^{no Follow} (Equation 5)

where n∈N and (d₁,s₁,d₂,s₂)∈NoFollowPairs. NoFollowPairs is a user defined set of pairs of day and shift indexes which indicates that a nurse cannot be assigned to both shifts. For example, if a nurse works a night shift (7 PM to 7 AM), the nurse should not be assigned to the day shift (7 AM to 7 PM) the following day.

Constraint 6: The assignment of nurses having a certain set of skills, certificates, etc. to particular shifts may be customized as being a hard constraint or a soft constraint as follows:

x_{n,d,s}≤isEligible_{n,s}+ [symbol] _{n,d,s}^{skill} (Equation 6)

where n∈N, d∈D, and s∈S. isEligible_{n,s} is defined by a user to be 1 if nurse n is eligible to work on shift s and 0 otherwise.

Constraint 7: The assignment of particular nurses with a certain seniority, title, etc. to particular shifts may be customized as being a hard constraint or a soft constraint as follows:

$${\sum }_{n ∈ N : i s C h a r g {e}_{n} is True} {x}_{n , d , s} \geq 1 - {ℓ}_{d , s}^{c h a r g e} \qquad ( Equation 7 )$$

where d∈D and s∈S. isChargeₙ is defined by a user to be True if nurse n has a particular seniority, title, etc. (e.g., if nurse n is a charge nurse) and False otherwise.

Constraint 8: A particular number of hours particular nurses are assigned may be customized as being a hard constraint or a soft constraint. The weekly working hours of a nurse are typically determined based on the contract of the nurse and paid time off taken by the nurse during the week. A full-time nurse typically works 36 hours per week whereas part-time and per diem nurses have different rules depending on the employer and the agreement type. Accordingly, general constraint rule is constructed that can be used to cover different scenarios that prorate the weekly working hours of a nurse by the time off requests. An index of a week w∈W is introduced, where W represents the set of weeks. The inputs associated with this constraint are: shiftHrsₛ representing the length of a shift s, workHrs_{n,w} representing the working hours of nurse n in week w, and PTOHrs_{n,w} representing the total time off hours of nurse n in week w. The constraints on a particular number of hours particular nurses are assigned are as follows:

$${\sum }_{d ∈ {D}_{w} , s ∈ S} ( shiftHR {S}_{s} \times {x}_{n , d , s} ) = workH r {s}_{n , w} - PTOHr {s}_{n , w} + {ℓ}_{n , w}^{w o r k H o u r s} \qquad ( Equation 8 )$$

where n∈N and w∈W and D_{w} is the set of days in week w. When this constraint is defined to be a soft constraint, the slack variable [symbol] _{n,k}^{workHours} can be positive or negative to respectively indicate under time and overtime for a nurse n.

Constraint 9: A minimum number of nurses assigned to particular shifts may be customized as being a hard constraint or a soft constraint. Shift coverage is one of the most important operational requirements for healthcare providers to ensure that a sufficient number of nurses are on duty to handle the patient load while maintaining quality of care and fiscal viability. The constraints for a minimum number of nurses assigned to particular shifts are as follows:

$${\sum }_{n ∈ N} {x}_{n , d , s} = r e q u i r e m e n {t}_{d , s} + {ℓ}_{d , s}^{c o v e r a g e} \qquad ( Equation 9 )$$

where d∈D and s∈S. requirement_{d,s} is the required number of nurses for shift s at day d. The slack variable [symbol] _{d,s}^{coverage} is a positive or negative integer representing a number of excess or missing nurses respectively. Due to the issues with retention and shortage of nurses, there will often be a negative deviation (i.e., a negative value of the slack variable [symbol] _{d,s}^{coverage}) between the ideal target number of assign nurses and the actual scheduled number of assigned nurses. Accordingly, it is important to distribute the deviations in the number of assigned nurses over days and shifts.

Constraint 10: The assignment of particular nurses to weekend shifts may be customized as being a hard constraint or a soft constraint. Weekend shift assignments are an important aspect of fair scheduling. Ideally, nurses are scheduled to work an equal number of weekend shifts across the scheduling period. Healthcare providers typically have differing rules in creating schedules with fair weekend shift allocations. There are two common rules from healthcare providers. The first rule is the assignment of nurses to a given number of weekend days for a given period, denoted by numWeekendₙ. The constraints for the assignment of nurses to a given number of weekend days for a given period are as follows:

$${\sum }_{d ∈ w e e k e n d , s ∈ S} {x}_{n , d , s} = n umWeeke n {d}_{n} + {ℓ}_{n}^{n umWeeke n d} \qquad ( Equation 10 )$$

where n∈N. weekend is the set of weekend days in the scheduling period. The second rule is the assignment of each nurse to weekend dates identified by that nurse. The constraints for the assignment of each nurse to weekend dates identified by that nurse are as follows:

$${\sum }_{{d ∈ D , d ∉ {workWeekend}_{n} , s ∈ S}_{d ∈ weekend}} {x}_{n , d , s} = {ℓ}_{n}^{workWeekend} \qquad ( Equation 11 )$$

where n∈N. workWeekendₙ is a set of work weekend dates identified by nurse n as being available to work. One or both of the rules may be applied as constraints for scheduling the nurses to shifts. The second rule when deployed as a hard constraint limits the possible work weekend dates of a nurse to the dates provided by that nurse. However, the assignment is not guaranteed on those dates. On the other hand, when the second rule is deployed as a soft constraint, the provided weekend dates can be considered as preferences. The soft constraint of the second rule is not combined with the assignment of nurses to their preferred shifts or days (constraint 11) as managers may want to prioritize weekend preferences over general preferences.

Constraint 11: The assignment of nurses to their preferred shifts or days may be customized as being a hard constraint or a soft constraint. A full-time nurse is typically required to work 36 hours a week, which corresponds to three or four shifts. As there are multiple shifts in a day, over a scheduling period a nurse can be assigned to different shifts such as a combination of different day and evening shifts. Although such assignments are possible, many nurses prefer certain days or shifts that they are assigned to and certain days or shifts that they are not assigned to.

When managers assign shifts, seniority and contract type (i.e., full-time vs. part-time) are typically considered. For example, if a senior and a non-senior nurse both prefer the same shift, it is likely that the manager will assign that shift to the senior nurse. This logic is incorporated by associating a weight for each day-shift combination for each nurse representing the desirability of a particular shift such that a positive or negative weight represents favorability or unfavorability of a shift respectively. The weight for each day-shift combination is denoted pref_{n,d,s}, representing the preference weight for nurse n, day d, and shift s. Different weighting schemes may be employed based on what is prioritized. In one example, a senior nurse's preferences are weighted by {−4, −3, 0, 3, 4} whereas a non-senior nurse's preferences are weighted by {−2, −1, 0, 1, 2}.

The weekly working hours of a nurse are determined by contract type and paid time off. If a nurse has an agreement for 36 working hours per week and uses 12 hours of paid time off, then their total working hours for the week are reduced to 24 hours. The total working hours for the scheduling period are defined as totalWorkHrsₙ, indicating the total working hours of nurse n through the scheduling period. maxScoreₙ is defined as the maximally achievable preference score for nurse n according to the following optimization problem:

$$max {\sum }_{d ∈ D , s ∈ S} {pref}_{n , d , s} \times shiftHr {s}_{s} \times {x}_{n , d , s}$$

$$s . t . {\sum }_{d ∈ D , s ∈ S} {shiftHrs}_{s} \times {x}_{n , d , s} \leq totalWorkH r {s}_{n}$$

where x_{n,d,s}∈{0,1} and ∀d∈D and s∈S. The deviation from this tight upper bound can be minimized for each nurse. However, the computation of this bound requires the solution of a knapsack problem, which is known to be NP-hard. To alleviate this issue, the linear programming relaxation of maxScoreₙ is solved. That is, x_{n,d,s}∈{0,1} is replaced with 0≤x_{n,d,s}≤1. Although this maximum score is not necessarily tight, it can be utilized as a target value for positive preferences.

The maxScoreₙ formulation of preferences has two goals. The first goal is to minimize the deviation from maxScoreₙ for each nurse for positive preferences to thereby assign nurses to their highly desired shifts. The deviation from maxScoreₙ is measured with a slack variable [symbol] ₙ^{posPref} for nurse n:

$${\sum }_{d ∈ D , s ∈ S} {\sum }_{{pref}_{n , d , s} > 0} {pref}_{n , d , s} \times shiftHr {s}_{s} \times {x}_{n , d , s} \geq {maxScore}_{n} - {ℓ}_{n}^{p o s Pref} \qquad ( Equation 12 )$$

The second goal is to minimize the negative preferences to thereby avoid assigning nurses to their undesirable shifts. The deviation of the negative preferences is measured by the slack variable [symbol] ₙ^{negPref} for nurse n:

$${\sum }_{d ∈ D , s ∈ S} {\sum }_{p r e {f}_{n , d , s} < 0} {pref}_{n , d , s} \times shiftHr {s}_{s} \times {x}_{n , d , s} = {ℓ}_{n}^{n e g Pref} \qquad ( Equation 13 )$$

The left-hand side of Equation 12 and Equation 13 respectively correspond to the sum of positive and negative weights of the assignment of nurse n. The slack variable [symbol] ₙ^{posPref} quantifies how far the assignment of nurse n is from the preferred shifts or days of nurse n. The slack variable [symbol] ₙ^{negPref} quantifies the assignment of unpreferred shifts or days of nurse n. The inequality (≥) rather than the equality (=) is used in the constraint of Equation 12 as overtime working assignments can make the constraint infeasible since the sum of assigned positive preferences can exceed maxScoreₙ.

Optimization: The slack variables associated with the soft constraints represents the deviation of the schedule from the soft constraints. For example, in the case of shift coverage, the slack variable indicates the number of missing or excess nurses for each shift, which represents the deviation in the assigned number of nurses from the target number (i.e., the soft constraint). The distribution of slack variables of the soft constraints across the determined schedule is important as high variance and the presence of outliers impacts the quality of the determined schedule. Balancing the slack variables across each the determined schedule may result in less variance and eliminates outliers, resulting in a higher quality schedule.

To simply this description, a single soft constraint is first considered together with one or more hard constraints that are not explicitly expressed for brevity. Let [symbol] ᵢ be a slack variable associated with a soft constraint, where i is an index or a set of indexes. The following cost function is considered for optimizing the slack variable:

$$min {\sum }_{i} f ( {ℓ}_{i} )$$

where ƒ(.) is the function penalizing the slack variable.

Conventionally, the cost function optimized by defining the cost function as a linear cost function and/or by minimizing the maximum deviation. However, such conventional approaches do not distribute or balance the slack variables across the determined schedule. For example, for the shift coverage example, such conventional approaches do not differentiate between the case where ten nurses are missing from one shift versus one nurse missing from ten shifts.

In one approach, the cost function is optimized by defining the cost function as a non-linear function (e.g., a quadratic) to take into account the differences in the distribution of slack variables. The resulting optimization problem is then a mixed-integer quadratic program (MIQP), which imposes a significant computational burden as compared to a mixed-integer linear program (MILP) for penalizing linear functions.

In accordance with one embodiment, the cost function is optimized by defining the cost function as a piecewise linear cost function approximating quadratic functions to thereby distribute deviations of the determined schedule from the soft constraints across the determined schedule with computational efficiency. Formally, given a set of k points, a₁<a₂< . . . <aₖ, a piecewise linear cost function pwƒ(.) is defined such that ƒ(aᵢ)=pwƒ(aᵢ) with breakpoints (a₁,ƒ(a₁)), (a₂,ƒ(a₂)), . . . , (aₖ,ƒ(aₖ)).

Lemma 1: Let ƒ(.) be a quadratic cost function whose domain χ is discrete and pwƒ(.) be a piecewise linear cost function whose function value is equivalent to ƒ(.) for every point in the domain. Then, minimizing pwƒ(.) over χ is equivalent to minimizing ƒ(.) over χ.

In the most general case, for Lemma 1 to hold, the quadratic cost function ƒ(.) is approximated by creating a piecewise linear cost function pwƒ(.) with breakpoints at every integer. However, such an approximation is computationally intractable. Accordingly, in one embodiment, a suitable number of breakpoints is selected for each constraint to determine a balanced schedule. For example, the number of breakpoints can be chosen to achieve a desirable computational time.

Considering now the multiple soft constraints with the hard constraints, a multi-objective optimization problem is defined. The multi-objective optimization problem considers one or more of the following slack variables: [symbol] _{n,w}^{workHours}, [symbol] _{d,s}^{coverage}, [symbol] n^{numWeekend}, [symbol] ₙ^{workWeekend}, [symbol] ₙ^{posPref}, [symbol] ₙ^{negPref}, [symbol] _{n,d}^{maxShifts}, [symbol] _{n,d}^{maxDays}, [symbol] _{n,d1,s1,d2,s2}^{noFollow}, [symbol] _{n,d,s}^{skill}, and [symbol] _{d,s}^{charge}, For each soft constraint, a single objective function is defined as:

$${F}_{n a m e} = {\sum }_{i} {f}_{n a m e} ( {ℓ}_{i}^{n a m e} )$$

where slack variables due to the same constraint are treated equally by applying the function ƒₙₐₘₑ.

In general, most of the objective functions are conflicting, particularly when there is a nurse shortage. In such cases, it is often impossible to find a solution where the all objective functions are minimized. In one embodiment, a schedule is determined by optimizing the multi-objective optimization problem by identifying a Pareto optimal solution.

Definition 1: Given k objective functions, F₁(x), F₂(x), . . . , Fₖ(x), a solution x* is Pareto optimal if and only if there exists no other solution x such that Fᵢ(x)<Fᵢ(x*), ∀i∈{1, . . . ,k}.

In a Pareto optimal assignment, a particular objective function cannot be improved without incurring additional losses in other objective functions. A Pareto optimal solution is the best solution of the optimization of a multi-objective optimization problem. For example, a Pareto optimal solution may be a solution where overtime hours for nurses cannot be reduced without losing coverage of shifts or to a solution where staff preferences will degrade if assignments are modified. In the nurse scheduling problem, assignments from the set of Pareto-optimal solutions that are non-dominating with respect to each other are of interest. A solution outside of this set will be sub-optimal for one or more objective functions.

The determined schedule is selected from the set of Pareto-optimal solutions, e.g., by the user. While it is desirable to identify all Pareto-optimal solutions, the identification of all Pareto-optimal solutions, known as finding the Pareto-front of the optimization problem, is known to be very hard for MILPs. As the multi-objective optimization problem is a MILP, which takes a long time even for a single instance of execution, it is not desirable to spend additional time finding all solutions where most of them are not used.

In accordance with one embodiment, two different approaches may be used to identify the Pareto-optimal solutions: lexicographic and weighted-sum. Both approaches preset different perspectives in configuring priorities for healthcare providers.

The Lexicographic approach is useful when a specific order of importance of the objective functions exist. The order can be represented as a one-to-one function from the rank to the objective. Let σ denote the one-to-one function from {1, . . . , k} to the set of the names (indexes) of the objectives where k is the number of the objectives. σ is defined so that the order of importance is reflected. For example, F_{σ(i)} has precedence over F_{σ(j)} if i<j. Lexicographic optimality is then defined according to Definition 2.

Definition 2: Given k objective functions F₁(x), F₂(x), . . . , Fₖ(x) and σ representing the order of importance of the objective functions, a solution x* is lexicographically optimal according to a if, for all solutions x, there exists i∈{1, . . . ,k} such that F_{σ(j)}(x*)=F_{σ(j)}(x) for j=1, . . . , i−1 and F_{σ(j)}(x*)≤F_{σ(i)}(x).

The multi-objective optimization problem can be lexicographically solved by sequentially solving each objective function based on the order of importance, together with the hard constraints. From the construction of the lexicographic approach, it can be seen that the resulting solution will belong to the set of Pareto optimal solutions. As the value of the object function obtained at a step i is added as a constraint for the next step i+1, the choice of the cost functions has a significant impact on the solution time. The use of quadratic cost functions would result in mixed-integer quadratically constrained programs that are harder to solve than MIQPs whereas the use of linear or piecewise linear functions does not alter the program type at hand.

In the weighted-sum approach, each objective of the multi-objective function is weighted and the multi-objective function is optimized (e.g., minimized) based on the weighs. Given weights w₁,w₂, . . . ,wₖ∈ [symbol] ⁺ corresponding to each objective function, the Pareto-optimal solutions are solved with a single objective function as follows:

$$min {\sum }_{i = 1}^{k} {w}_{i} {F}_{i} ( x )$$

The lexicographic approach is useful when there is an absolute preference among the objective functions and it is straightforward for users to configure and interpret the returned assignments. This simplicity makes it more explainable and intuitive as to why certain requirements are not satisfied as much as others that are ranked higher. As the lexicographic approach finds the optimum values of one cost function at a time, it eliminates Pareto optimal solutions that can trade-off imperfections between constraints. For example, it will not allow solutions where there are minor deficiencies in coverage in exchange for better weekend assignments. Also each distinct ordering of objective functions might not necessarily correspond to different Pareto optimal solutions as the optimization problem can be too constrained to be sensitive to the order of the least important objectives. Another limitation of the lexicographic method is when there is no clear preference between certain objective functions such as the shift coverage constraint and the weekend shift assignment constraint. Unlike the lexicographic approach, the weighted-sum approach can output Pareto optimal solutions that are not necessarily lexicographically optimal.

In one embodiment, optimization agents that encapsulate specific choices in a predetermined way are provided. The optimization agents can combine lexicographic and weighted solution schemes to make the optimization of multiple objectives more intuitive to the user. The optimization agents can be created at the time of deployment based on feedback from scheduling managers. In one example, the optimization agents include a target time aware optimization agent that performs an optimization that equally values the weekly target working hours and the number of weekend days constraints above the coverage constraint and followed all other constraints. In another example, the optimization agents include a target time and census aware optimization agent that performs an optimization that equally values the weekly target working hours and the coverage constraints above the number of weekend days constraint and followed by all other constraints. Other optimization agents are also contemplated, e.g., based on the needs of a unit or medical provider.

At step 106 of FIG. 1, the determined schedule is output. For example, the determined schedule can be output by displaying the determined schedule on a display device of a computer system, storing the determined schedule on a memory or storage of a computer system, or by transmitting the determined schedule to a remote computer system.

Embodiments described herein were experimentally validated using synthetically generated requirements or constraints with batch testing at various resource levels. A general constraints generator was implemented that is flexible to simulate different numbers of nurses, days, shifts, shift lengths, cover requests, paid time off requests, unavailabilities, working hours, pre-assignments, and preferences. Constraints were generated for 50 nurses, 28 days, and 3 shifts per day that was referred to as morning, evening, and night shifts. To simulate realistic cover requests, for each shift type, the cover number is generated from a Poisson distribution with mean {8, 7, 5} respectively, and the generated number is capped to be within ±20% of the mean value to prevent outliers. For each nurse, two favorite days, one weakly desired day, and one undesired day was assigned that repeated for every week in the 28-day scheduling window. The favorite days have preference weight p randomly drawn from the integer set {2, 3, 4} whereas the weakly desired day has a weight p−1 and the undesired day has weight −p. In assigning favorite and non-favorite days and shifts to nurses, it is assumed that certain days and shifts would be more desirable than others. To that end, the favorite days were drawn from a distribution where mid-week days are more preferable to weekend days. In assigning favorite shifts, it is assumed the discrete probability distribution {0.45, 0.35, 0.2} associated with each shift.

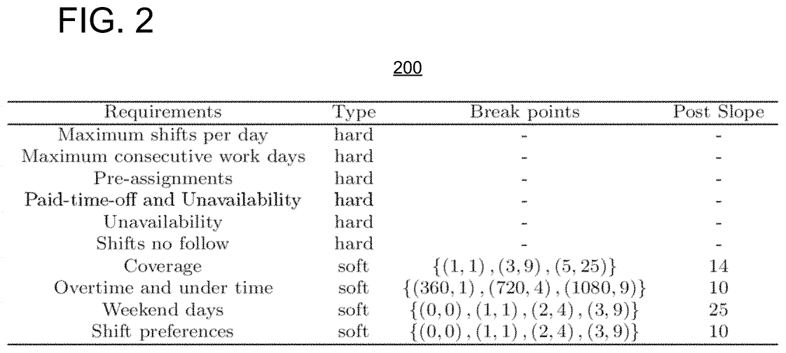

In the experiments with synthetically generated data, a subset of the constraints or requirements summarized in table 200 of FIG. 2 was used together with breakpoints for the piecewise linear functions. It is noted that the breakpoints are not extensively defined for piecewise linear cost functions, as this requires using a large number of breakpoints (see Lemma 1) and hence the results obtained by the piecewise linear and quadratic approaches may not be equivalent.

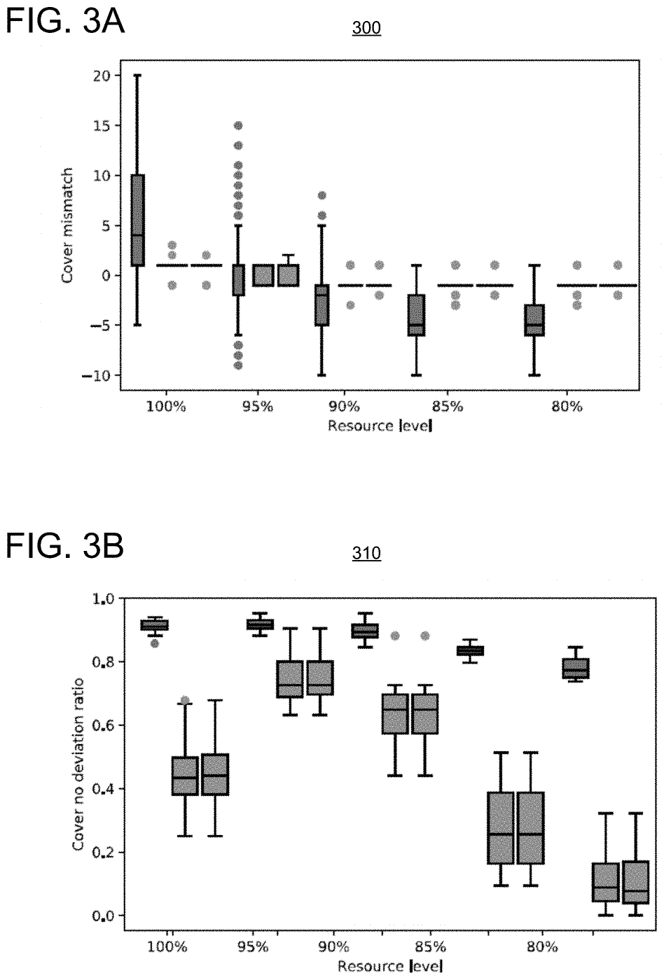

The first experiment focused on the distribution of deviation from requested coverage when no nurse is allowed to have over time or under time such that they work their prorated weekly working hours. To achieve this behavior, the lexicographic solution was applied with the weekly working hours set to top priority followed by the cover requests. Such a lexicographic order guarantees that the working hours of the nurses are not violated to no incur cost. FIG. 3A shows a graph 300 illustrating the distribution of excess or missing number of nurses for each shift across resource levels. In graph 300, three box plots are shown for each resource level corresponding to linear, piecewise linear, and quadratic approaches, from left to right. When the resource levels are sufficient to fill the shift coverage requests, it is observed that the linear approach results in shifts with high excess and high deficit. On the other hand, piecewise linear and quadratic approaches consistently achieved one extra nurse per shift with a few exceptions. This behavior is expected as the linear approach is indifferent to the balance of the slack variables lᵢ whereas the piecewise linear and quadratic approaches incur a higher cost if slack variables grow in magnitude. As the resource levels shrink, the linear approach shows similar behavior with deficient where there are shifts with a high number of missing nurses. Even with high shortage levels, piecewise linear and quadratic approaches can maintain the shortages at a uniform level across shifts. FIG. 3B shows a graph 310 illustrating the ratio of shifts that have no deviation from their target number of nurses. In graph 310, three box plots are shown for each resource level corresponding to linear, piecewise linear, and quadratic approaches, from left to right. The piecewise linear and quadratic approaches have a lower no-deviation ratio as they strive to have a balanced distribution of excess and missing nurses. If nurse hours are abundant compared to the number of shift hours to be filled, such as the case with 100% resources, many shifts are added one or two extra nurses. When the nurse hours are limited, many shifts have one or two missing nurses. This behavior is key to having balanced schedules and results in low no-deviation ratio. The linear approach maintains a high no-deviation ratio at the expense of having shifts that have very unbalanced nurse coverage that can hamper operations and patient care.

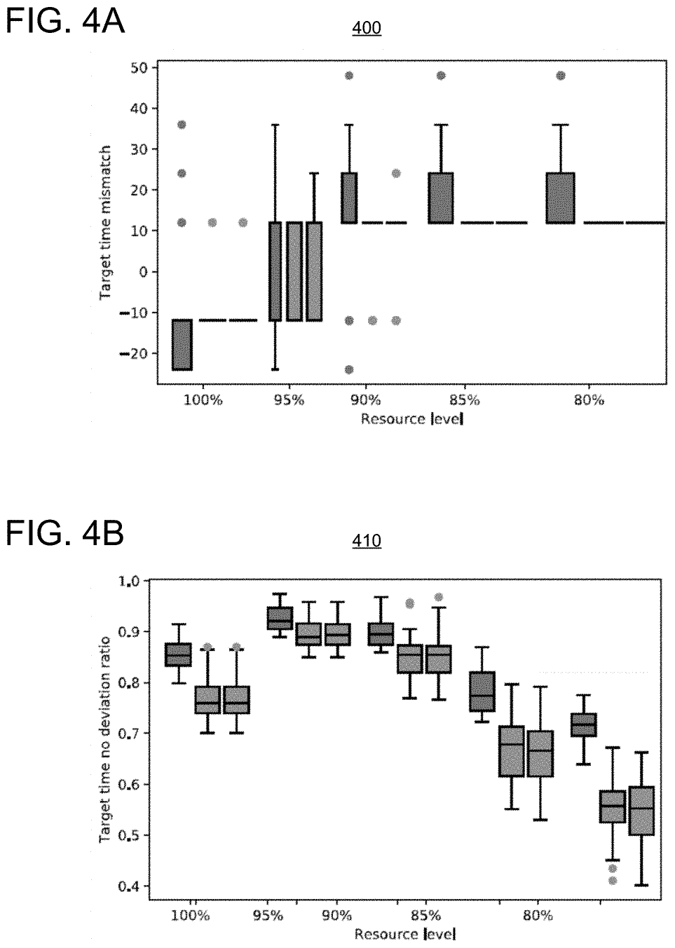

The second experiment observed the deviation in weekly working hours when the shift coverage requirement was prioritized over every other objective followed by the weekly working hours requirement. In this case, the coverage requirements will be fulfilled at the expense of the weekly working hours. FIG. 4A shows a graph 400 illustrating the distribution of deviation from target working hours. In graph 400, three box plots are shown for each resource level corresponding to linear, piecewise linear, and quadratic approaches, from left to right. When there is no resource shortage, the linear method results in schedules with up to 24 hours missing and 36 hours of overtime for some nurses. As resource levels get tighter, the number of nurses having overtime increases for all approaches, but the piecewise linear and quadratic approaches are able to maintain constant overtime. FIG. 4B shows a graph 410 illustrating the ratio of nurses that have no deviation from their target working hours. In graph 410, three box plots are shown for each resource level corresponding to linear, piecewise linear, and quadratic approaches, from left to right. Similar to the coverage of shifts example, piecewise linear and quadratic approaches have a lower no-deviation ratio as they strive to have balanced over and undertime for every nurse in the schedule.

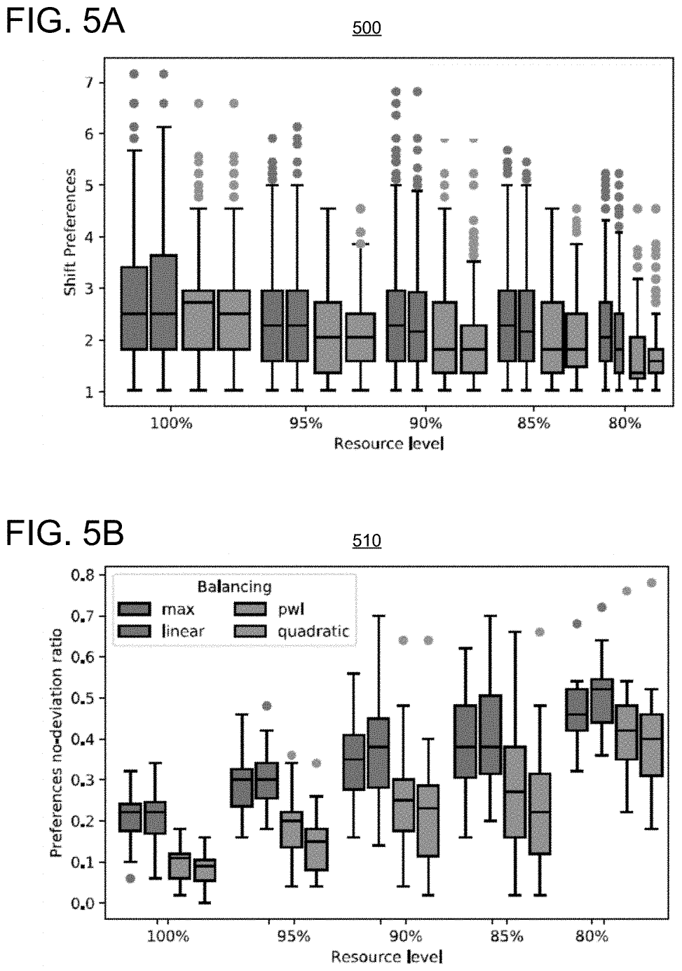

The third experiment observed the preference balancing for the assignment of nurses to their preferred shifts or days (constraint 11). Compared to individually maximizing preferred shifts and minimizing non-preferred shifts, embodiments described herein compute a surrogate maximum achievable score and penalizes the deviation from this score for each nurse. Similar to the previous experiments, multiple instances and resource levels were used to assess how preferences are distributed. In this setup, the coverage requirement was placed as the top priority followed by staff preferences. The deviation from the maximum achievable score was measured after the assignments. It is noted that preference allocation naturally results in conflicts especially when highly favored days and shifts form clusters as there is only a limited number of nurses needed for each shift. Therefore, it might never be possible to match the maximum score for each nurse. However, fair distribution of these highly preferred days and shifts can be achieved. In the experiments, the three balance-based methods and the naïve maximization of individual scores approach were compared across multiple resources. A deviation of less than one is left out as this difference can be due to using a surrogate for the maximum achievable score. FIG. 5A shows a graph 500 illustrating a deviation from the surrogate maximum achievable score. FIG. 5B shows a graph 510 illustrating no-deviation ratio. In graph 500, four box plots are shown for each resource level corresponding to linear, piecewise linear, quadratic and maximization approaches, from left to right. When there is no nurse shortage (100% resource level), it is hard to assign every nurse such that they attain their maximum achievable score, as many nurses will work under time. In this case, the deviations from the maximum achievable score can be high. When there is a serious nurse shortage, due to less competition and overtime work, nurses will possibly have more of their preferred shift assigned. In all cases, it is observed that the maximization approach results in the largest variation in the deviations from the maximum achievable scores. This behavior is not desired as this will result in the unfair assignment of preferred shifts. The quadratic and piecewise linear balancing approaches perform better than the linear deviation minimization method and they result in lower variance. The no-deviation ratios of preferences complement this behavior.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1. Certain steps or functions of the methods and workflows described herein, including one or more of the steps or functions of FIG. 1, may be performed by a server or by another processor in a network-based cloud-computing system. Certain steps or functions of the methods and workflows described herein, including one or more of the steps of FIG. 1, may be performed by a client computer in a network-based cloud computing system. The steps or functions of the methods and workflows described herein, including one or more of the steps of FIG. 1, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

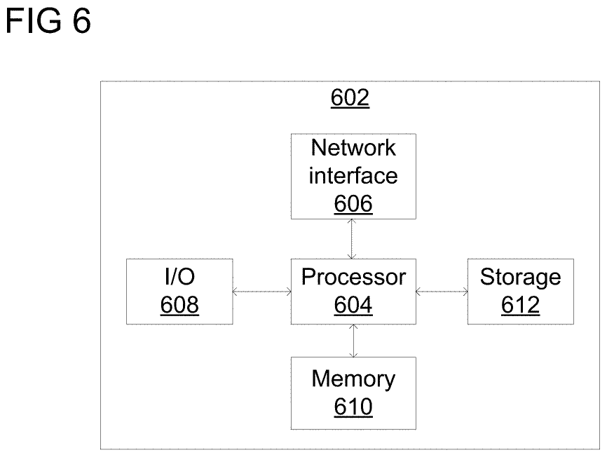

A high-level block diagram of an example computer 602 that may be used to implement systems, apparatus, and methods described herein is depicted in FIG. 6. Computer 602 includes a processor 604 operatively coupled to a data storage device 612 and a memory 610. Processor 604 controls the overall operation of computer 602 by executing computer program instructions that define such operations. The computer program instructions may be stored in data storage device 612, or other computer readable medium, and loaded into memory 610 when execution of the computer program instructions is desired. Thus, the method and workflow steps or functions of FIG. 1 can be defined by the computer program instructions stored in memory 610 and/or data storage device 612 and controlled by processor 604 executing the computer program instructions. For example, the computer program instructions can be implemented as computer executable code programmed by one skilled in the art to perform the method and workflow steps or functions of FIG. 1. Accordingly, by executing the computer program instructions, the processor 604 executes the method and workflow steps or functions of FIG. 1. Computer 602 may also include one or more network interfaces 606 for communicating with other devices via a network. Computer 602 may also include one or more input/output devices 608 that enable user interaction with computer 602 (e.g., display, keyboard, mouse, speakers, buttons, etc.).

Processor 604 may include both general and special purpose microprocessors, and may be the sole processor or one of multiple processors of computer 602. Processor 604 may include one or more central processing units (CPUs), for example. Processor 604, data storage device 612, and/or memory 610 may include, be supplemented by, or incorporated in, one or more application-specific integrated circuits (ASICs) and/or one or more field programmable gate arrays (FPGAs).

Data storage device 612 and memory 610 each include a tangible non-transitory computer readable storage medium. Data storage device 612, and memory 610, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 608 may include peripherals, such as a printer, scanner, display screen, etc. For example, input/output devices 608 may include a display device such as a cathode ray tube (CRT) or liquid crystal display (LCD) monitor for displaying information to the user, a keyboard, and a pointing device such as a mouse or a trackball by which the user can provide input to computer 602.

Any or all of the systems and apparatus discussed herein may be implemented using one or more computers such as computer 602.

One skilled in the art will recognize that an implementation of an actual computer or computer system may have other structures and may contain other components as well, and that FIG. 6 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
receiving a plurality of constraints for scheduling individuals to shifts, the plurality of constraints comprising one or more hard constraints and one or more soft constraints;
determining a schedule assigning the individuals to the shifts that 1) satisfies the one or more hard constraints and 2) distributes deviations from the one or more soft constraints across the determined schedule; and
outputting the determined schedule.

2. The computer-implemented method of claim 1, wherein the one or more soft constraints comprises preferences for desirable and undesirable shifts or days for one or more of the individuals, and determining a schedule assigning the individuals to the shifts that 1) satisfies the one or more hard constraints and 2) distributes deviations from the one or more soft constraints across the determined schedule comprises:
determining a maximally achievable preference score for the one or more individuals for the preferences; and
minimizing a deviation of the determined schedule from the maximally achievable preference score for the desirable shifts or days and minimizing a deviation of the determined schedule from the undesirable shifts or days.

3. The computer-implemented method of claim 1, wherein determining a schedule assigning the individuals to the shifts that 1) satisfies the one or more hard constraints and 2) distributes deviations from the one or more soft constraints across the determined schedule comprises:
optimizing one or more slack variables representing deviations of a schedule from the one or more soft constraints.

4. The computer-implemented method of claim 3, wherein optimizing one or more slack variables representing deviations of a schedule from the one or more soft constraints comprises:
defining one or more cost functions for optimizing the one or more slack variables as one or more piecewise linear cost functions.

5. The computer-implemented method of claim 1, wherein determining a schedule assigning the individuals to the shifts that 1) satisfies the one or more hard constraints and 2) distributes deviations from the one or more soft constraints across the determined schedule comprises:
optimizing a multi-objective optimization problem to identify a solution for each objective of the multi-objective optimization problem; and
selecting the determined scheduled from the identified solutions.

6. The computer-implemented method of claim 5, wherein optimizing a multi-objective optimization problem to identify a solution for each objective of the multi-objective optimization problem comprises:
lexicographically solving the multi-objective optimization problem by assigning an order of importance to each objective of the multi-objective optimization problem and sequentially solving each objective based on the assigned order of importance.

7. The computer-implemented method of claim 5, wherein optimizing a multi-objective optimization problem to identify a solution for each objective of the multi-objective optimization problem comprises:
weighting each objective of the multi-objective optimization problem; and
optimizing the multi-objective optimization problem based on the weights.

8. The computer-implemented method of claim 1, wherein the plurality of constraints comprises one or more of:
an assignment of one or more of the individuals to one or more of the shifts;
one or more of the shifts that one or more of the individuals cannot be assigned;
a maximum number of shifts in a day that one or more of the individuals can be assigned;
a maximum number of consecutive days one or more of the individuals can be assigned a shift;
two or more succeeding shifts that one or more of the individuals cannot be assigned a shift;
an assignment of one or more of the individuals having a certain set of skills or certificates to one or more of the shifts;
an assignment of one or more of the individuals with a certain seniority or title to one or more of the shifts;
a number of hours one or more of the individuals are assigned;
a minimum number of the individuals assigned to one or more of the shifts;
an assignment of one or more of the individuals to weekend shifts; and
an assignment of one or more of the individuals to their preferred shifts or days.

9. The computer-implemented method of claim 1, wherein the individuals are nurses.

10. An apparatus comprising:
means for receiving a plurality of constraints for scheduling individuals to shifts, the plurality of constraints comprising one or more hard constraints and one or more soft constraints;
means for determining a schedule assigning the individuals to the shifts that 1) satisfies the one or more hard constraints and 2) distributes deviations from the one or more soft constraints across the determined schedule; and
means for outputting the determined schedule.

11. The apparatus of claim 10, wherein the one or more soft constraints comprises preferences for desirable and undesirable shifts or days for one or more of the individuals, and the means for determining a schedule assigning the individuals to the shifts that 1) satisfies the one or more hard constraints and 2) distributes deviations from the one or more soft constraints across the determined schedule comprises:
means for determining a maximally achievable preference score for the one or more individuals for the preferences; and
means for minimizing a deviation of the determined schedule from the maximally achievable preference score for the desirable shifts or days and minimizing a deviation of the determined schedule from the undesirable shifts or days.

12. The apparatus of claim 10, wherein the means for determining a schedule assigning the individuals to the shifts that 1) satisfies the one or more hard constraints and 2) distributes deviations from the one or more soft constraints across the determined schedule comprises:
means for optimizing one or more slack variables representing deviations of a schedule from the one or more soft constraints.

13. The apparatus of claim 12, wherein the means for optimizing one or more slack variables representing deviations of a schedule from the one or more soft constraints comprises:
means for defining one or more cost functions for optimizing the one or more slack variables as one or more piecewise linear cost functions.

14. The apparatus of claim 10, wherein the means for determining a schedule assigning the individuals to the shifts that 1) satisfies the one or more hard constraints and 2) distributes deviations from the one or more soft constraints across the determined schedule comprises:
means for optimizing a multi-objective optimization problem to identify a solution for each objective of the multi-objective optimization problem; and
means for selecting the determined scheduled from the identified solutions.

15. A non-transitory computer readable medium storing computer program instructions, the computer program instructions when executed by a processor cause the processor to perform operations comprising:
receiving a plurality of constraints for scheduling individuals to shifts, the plurality of constraints comprising one or more hard constraints and one or more soft constraints;
determining a schedule assigning the individuals to the shifts that 1) satisfies the one or more hard constraints and 2) distributes deviations from the one or more soft constraints across the determined schedule; and
outputting the determined schedule.

16. The non-transitory computer readable medium of claim 15, wherein determining a schedule assigning the individuals to the shifts that 1) satisfies the one or more hard constraints and 2) distributes deviations from the one or more soft constraints across the determined schedule comprises:
optimizing a multi-objective optimization problem to identify a solution for each objective of the multi-objective optimization problem; and
selecting the determined scheduled from the identified solutions.

17. The non-transitory computer readable medium of claim 16, wherein optimizing a multi-objective optimization problem to identify a solution for each objective of the multi-objective optimization problem comprises:
lexicographically solving the multi-objective optimization problem by assigning an order of importance to each objective of the multi-objective optimization problem and sequentially solving each objective based on the assigned order of importance.

18. The non-transitory computer readable medium of claim 16, wherein optimizing a multi-objective optimization problem to identify a solution for each objective of the multi-objective optimization problem comprises:
weighting each objective of the multi-objective optimization problem; and
optimizing the multi-objective optimization problem based on the weights.

19. The non-transitory computer readable medium of claim 15, wherein the plurality of constraints comprises one or more of:
an assignment of one or more of the individuals to one or more of the shifts;
one or more of the shifts that one or more of the individuals cannot be assigned;
a maximum number of shifts in a day that one or more of the individuals can be assigned;
a maximum number of consecutive days one or more of the individuals can be assigned a shift;
two or more succeeding shifts that one or more of the individuals cannot be assigned a shift;
an assignment of one or more of the individuals having a certain set of skills or certificates to one or more of the shifts;
an assignment of one or more of the individuals with a certain seniority or title to one or more of the shifts;
a number of hours one or more of the individuals are assigned;
a minimum number of the individuals assigned to one or more of the shifts;
an assignment of one or more of the individuals to weekend shifts; and
an assignment of one or more of the individuals to their preferred shifts or days.

20. The non-transitory computer readable medium of claim 15, wherein the individuals are nurses.
